(19) United States
(12) Patent Application Publication
(10) Pub. No.: US 2004/0053319 A1
(43) Pub. Date: Mar. 18, 2004

(54) Isolation of genetic molecules from a complex biological constrauct for use in genetic expression analysis

(76) Inventors: Diana R. McWilliams, Bonne Terre, MO (US); Pamela A. De Ciechi, O'Fallon, MO (US)

Correspondence Address: Carol M. Nielsen, GARDERE WYNNE SEWELL LLP, Patent Section (H), 1601 Elm Street, Suite 3000, Dallas, TX, 75201, US

(21) Appl. No.: 10/661,729

(22) Filed: Sep. 12, 2003

Related U.S. Application Data
(60) Provisional application No. 60/411,175, filed on Sep. 17, 2002.

(51) Int. Cl. C12Q 1/68

(52) U.S. Cl. 435/6

(57) ABSTRACT

A method and apparatus for the extraction and isolation of genetic molecules such as DNA, RNA, mRNA, rRNA or tRNA from an animal for use in the analysis of genetic expression is provided. The present method and apparatus of the subject invention are particularly useful in high throughput, automated analysis of genetic molecular levels and function.

### CROSS-REFERENCE TO RELATED APPLICATIONS

The present application claims priority under Title 35, United States Code, § 119(e)(1) of U.S. Prov. Pat. App. Ser. No. 60/411,175, filed Sep. 17, 2002.

### BACKGROUND

Currently at the forefront of gene expression research are experiments and analysis to determine the effect of genetic history and various biological stimuli on the overall gene expression patterns of different cells. Particularly, in vivo experimentation of pharmacological products mandates an accurate analysis of the cellular function and gene expression to determine efficacy and safety. The existence or nonexistence of a genomic reaction may be indicative of the success or failure of the drug product and the risk exposure to the patient by administering such drug. Therefore, the degradation of RNA as well as the change of transcriptional levels of specific RNA in such gene expression analysis must be avoided.

RNA has the ability to change transcription level very quickly, so quickly that minutes may actually change the level of certain transcriptional RNAs. The cell biologist and other experienced researchers who initiate an experiment and analyze the results of gene expression must collect and analyze animal tissues as quickly as possible, beginning at the time the animal is euthanized and the organs harvested.

In harvesting the organs, animal tissue is typically flash frozen in liquid nitrogen and shipped to a laboratory via dry ice. Current protocols have been directed to preserving RNA after it is liquefied via the lysis solution itself and do little to prevent degradation prior to lysis and liquefaction. For example, RNALater made by Ambion requires the researcher to mince the tissue into small pieces so the solution can more easily penetrate the tissue.

However, specific analysis of cellular function requires release of the intracellular contents, a complex and time-consuming process. For example, the slowest and most time consuming part of the molecular analysis of RNA may be the actual pulverization of tissue into a liquefied or frozen pulverized form prior to RNA isolation and the reverse transcriptase (“RT”) and polymerase chain reaction (“PCR”) analysis.

It is essential that cell lysis be accomplished as rapidly as possible to prevent the RNA from being degraded by ribonuclease. Ribonuclease is any enzyme that catalyzes the cleavage of nucleotides in RNA. Ribonuclease (also known as “RNase”) catalyze the hydrolysis of phosphate ester linkages in ribonucleic acid. Each RNase has specificity for a different cleavage site. For example, RNase A is a digestive enzyme secreted by the pancreas that hydrolyses phosphodiester bonds in nucleotide complexes. Other RNase are active at the cellular level, for instance in modifying transfer RNA (“tRNA”) and ribosomal RNA (“rRNA”) after transcription.

Endonuclease-mediated mRNA decay is one important pathway of regulating mRNA turnover rate although the mechanism involved in targeting substrate mRNA to degradation is largely unknown. Most cellular and viral RNA undergo one or more highly specific processing reactions to attain their mature, functional form. In addition, all RNA ultimately is degraded to mononucleotides that provide precursors for new RNA synthesis.

RNA processing and degradation reactions are key in controlling gene expression and establishing cell phenotypes. For example, rapid mRNA degradation coupled to transcriptional switches allows the cell to rapidly reprogram its pattern of gene expression and change in its phenotype. As catalysts of RNA cleavage, ribonuclease act at the crossroads of transcription and translation. Ribonuclease are cytotoxic under certain conditions and problematic in the analysis of gene expression because cleaving RNA renders indecipherable its encoded information.

Hence, degradation of DNA and RNA as a result of endogenous and contaminating nucleases during isolation of nucleic acids from biological samples is of particular concern. When quantitative analysis of endogenous mRNA levels is the focus of the experimental analysis, it is critical the RNA remain high quality. Each sample has very little RNA and RNA is highly susceptible to rapid degradation.

Traditional methods in molecular biology generally work on a one gene per one experiment basis, which means that the throughput is very limited and the overall picture of gene function is hard to obtain. In the past several years, however, new technology referred to as the “DNA microarray” which is an orderly arrangement of samples has been of tremendous interest to biologists and researchers alike. Use of a genetic array provides a medium for matching known and unknown DNA samples based on base-pairing rules and automating the process of identifying the unknowns. An array experiment can make use of common assay systems such as microplates or standard blotting membranes, and can be created by hand or utilize robotics to deposit the sample.

In the analysis of gene expression, simultaneous extraction and isolation of genetic molecules from a variety of different tissue samples is preferred and often necessary. In addition, overall gene expression over a large structural region or functional system of an animal is also desirable. Such samples are extremely heterogeneous, containing a multitude of tissues requiring different levels of processing for nucleic acid release. Extraction and isolation of nucleic acids from individual components may be performed and the resulting samples left unique or pooled. However, known methodologies to accomplish RNA/DNA isolation are extremely time consuming. In such cases, the step of liquefying tissue must be rapid but not so disruptive that chemical changes and/or molecular degradation result from the lysis procedure itself.

A number of different methods have been developed in the art including disruption by mechanical force, application of high pressure, ultrasonication, chemical extraction, enzymatic digestion, and heat disruption. These methods vary in their effectiveness in disrupting tissue and are limited to a single animal organ or tissue type.

Lysis methods are typically tissue specific or designed for cellular disruption of a particular tissue. Another method, heat disruption, can inactivate enzymes by causing protein denaturation. Ultrasonication is not suitable for the isolation of genomic DNA since genomic DNA is very easily sheared by the forces produced thereby. A number of devices and protocols for high throughput, automated analysis of gene expression levels are in development. However, high throughput protocols for rapid and efficient release, extraction and isolation of RNA/DNA from biological samples remain limited.

In a busy laboratory, a high quality yield of RNA is often compromised for obtaining the maximal yield, that is, isolating enough total RNA to satisfy the needs of the microarray or orderly arrangement of samples. In addition, if different tissue samples must be retrieved and analyzed concurrently, the amount and quality of the RNA yield may be compromised. When the expression of a multitude of genetic molecules is tested at the same time, rapid lysis and liquefaction of the tissue source is critical.

Hence, in order to fully assess and test gene expression patterns in an animal, a need exists for a method and device that avoids the use of sampling a particular tissue and allows rapid and efficient extraction and isolation of genetic molecules such as DNA, RNA, mRNA, rRNA and tRNA from several animal tissues simultaneously while minimizing the risk of degradation of transcript levels.

### SUMMARY OF THE INVENTION

The subject invention is a method and apparatus for the extraction and isolation of genetic molecules such as DNA, RNA, mRNA, rRNA or tRNA from an animal for use in the analysis of genetic expression. The present method and apparatus of the subject invention are particularly useful in high throughput, automated analysis of genetic molecular levels and function. The present invention avoids the current need for sampling a single tissue type and the added steps of obtaining one particular type of tissue. By extracting and isolating genetic molecules from an entire limb or other complete anatomical culture, the risk of sample contamination and mRNA degradation or RNA change after the animal is euthanized is significantly reduced.

The method for extraction and isolation of genetic molecules for use in the analysis of genetic expression comprises the steps of liquefying or pulverizing a complex biological construct into solution or powder having complete and uncontaminated genetic molecules, transferring the solution or powder to a Taqman assay or a microarray, and determining gene expression and/or function.

### BRIEF DESCRIPTION OF SEVERAL VIEWS OF THE DRAWINGS

For better understanding of the invention and to show by way of example how the invention may be carried into effect, reference is now made to the detail description of the invention along with the accompanying figures in which corresponding numerals in the different figures refer to corresponding parts and in which:

FIG. 1 depicts a cross-sectional view of a sealed chamber with grinding element.

FIG. 2 depicts a perspective view of a sealed chamber with liquefying/pulverizing component.

FIG. 3 depicts a perspective view of a freezer mill suitable for use in connection with the subject invention.

FIG. 4 depicts a perspective view of a mixer mill suitable for use in connection with the subject invention.

FIG. 5 depicts a perspective view of a tissue crusher suitable for use in connection with the subject invention.

FIG. 6 is a graphic depiction of the relative transcription levels of Gene A of Example 3.

FIG. 7 is a graphic depiction of the relative transcription levels of Gene B of Example 3.

FIG. 8 is a graphic depiction of the relative transcription levels of Gene C of Example 3.

### DETAILED DESCRIPTION

The subject invention is a method and apparatus for the extraction and isolation of genetic molecules such as DNA, RNA, mRNA, rRNA or tRNA from an animal for use in the analysis of genetic expression. The present method and apparatus of the subject invention are particularly useful in high throughput, automated analysis of genetic molecular levels and function.

The method for extraction and isolation of genetic molecules for use in the analysis of genetic expression comprises the steps of liquefying or pulverizing a complex biological construct into solution or powder having complete and uncontaminated genetic molecules, transferring the solution to a Taqman assay or microarray, and determining gene expression and/or function. The apparatus for performing the method comprises a chamber fitted with a component that will fracture the complex biological construct and ruptures it cells. The apparatus also comprises a means for applying mechanical force to the chamber whereby the component will rupture the cells releasing genetic molecules into solution.

Liquefaction and liquefy refer to any process in which a solid or solid suspension is homogenized so that material appears to be a liquid. The material may, in fact, be either a solution, or suspension of particles of submicroscopic size.

Genetic molecules as referred to herein include genomic DNA, episomal DNA, messenger RNA (“mRNA”), heteronuclear RNA (“hnRNA”), transfer RNA (“tRNA”) and ribosomal RNA (“rRNA”).

A complex biological construct as used herein may be any portion of an animal having more than one tissue type. The complex biological construct may comprise an entire limb of animal or other gross anatomical structure such as appendages, organs, collection of organs, or organ systems. The complex biological construct may include, but are not limited to, hair, bone, blood, blood vessels, muscles, connective tissue, cartilage, nerve, bone marrow, epithelium, and adipose tissues.

A complex biological construct useful in the method of the present invention may contain many of the tissues that make up an animal. The body of the animal, also referred to as the organism, can be understood at seven related structural levels: chemical, organelle, cellular, tissue, organ, organ system and finally the entire body or organism, or a discrete portion or part of it. A tissue by definition is a group of cells with similar structure and function. An organ is composed of two or more tissue types that perform one or more common function. The organ system is a group of organs classified as a unit because of a common function or set of functions. The complex biological construct of the subject invention, however, will contain several types of tissue potentially having a diversity of function and may potentially contain numerous cell types. For example, there are over 200 types of cells in the human body assembled into a variety of tissue types.

The four primary tissue types are epithelial, connective, muscular, and nerve. Each primary tissue type has several subtypes. Epithelial tissues include membranous and glandular. Connective tissues include connective tissue proper and specialized connective tissue. The three subtypes of muscle tissue are skeletal, cardiac and smooth. The nerve cells are specialized form of communication and are composed of a network of neurons among supporting glial cells. The epithelia and connective tissues are the most abundant and diverse of the four tissue types and are components of every organ in the human body.

In epithelial tissues, cells are tightly bound together into sheets called epithelia. The epithelia tissue consists primarily of cells, and it is cells rather than the matrix that bear most of the mechanical stress. Epithelial cell sheets line all the cavities and free surfaces of the body and the specialized junctions between the cells enable these sheets to form barriers to the movement of water, solutes, and cells from one body compartment to another. Epithelial sheets almost always rest on a supporting bed of connective tissue which may attach them to other tissues such as muscle that do not themselves have either strictly epithelial or strictly connective tissue organizations.

There are many specialized types of epithelia. However, whereas epithelia may be specialized for unique functions in an organ system, they all have some features in common. First, the cells are apposed to one another and line a surface. Second, they sit on a layer of fine filaments, called a “basal lamina”. Collectively these layers form a boundary between the external environment and the remainder of the organ. Thus, at the most basic level, epithelia are organized to control movement of substances into and out of that organ.

In addition, a stratified epithelium may provide more protection to the organ against friction and the like since the outer layers of the cells could be sloughed off as the epithelium encounters friction. Simple epithelia regulate transport through the epithelial cells by membrane transport proteins, endocytosis and special barrier junctions.

The shape of the cell facilitates determination of its function. For example, flattened, scale-like cells (referred to as squamous) may be seen in one layer (simple) or in multiple layers (stratified). If these cells are in a single layer, they provide minimal protection, but often provide more opportunity for passive transport of substances across the cell. For example, the capillary wall is where epithelial cells provide the surface area for transport of gases and other molecules. If squamous cells are in a stratified epithelium, they are often designed for protection against invasion or friction. They have desmosomes (junctions) and can be sloughed off and replaced rapidly.

Epithelia that are cube shaped are called, appropriately, “cuboidal”. Often these epithelia have specialized junctions and transport processes that control movement of substances from one side to the other. Sometimes they are secretory. Thus, the taller the cell, the more active it may be in terms of regulated transport. This is particularly true of the tallest epithelial cells, the columnar cells. Shaped like a column, these cells often have very different, specialized surfaces designed to protect the barrier and transport into the cell and then out of the cell. Some epithelial cells, such as the thyroid, become taller as they secrete more.

Finally, there are the transitional epithelium in bladder or ureter that are not classified. This epithelium may have cells that are squamous and even columnar. It is definitely multilayered. It also may distend so that it looks like it is only 2-3 cellular layers.

Various types of cells in the epithelium perform different function. Absorptive cells in epithelial have numerous hair-like microvilli projecting from their free surface to increase the area for adsorption. Ciliated cells have cilia in their free surface that beat in synchrony to move substances over epithelial sheet. Secretory cells are found in most epithelial layers and exude substances onto the surface of the cell sheet.

Connective tissues are classified as connective tissue proper and specialized connective tissue. The specialized connective tissue includes cartilage, bone, and blood. Connective tissue proper has a matrix comprising numerous fibers that are collagenous, elastic, or reticular (branched). The connective tissue proper includes dense connective tissue and loose connective tissue. The loose or areolar connective tissue has an intercellular matrix widely distributed in the body and found most readily beneath the skin and superficial fascia (fatty connective tissue) separating muscles, in all potential spaces, and beneath the epithelial lining in lamina propria of the digestive system. The web-like tissue binds cells and organs together but permits the cells and organs to move, as necessary in relation to each other. Loose connective tissue is composed of a large amount of amorphous ground substance whose consistency varies from liquid to gel, allowing cells to move around freely and other structures such as blood vessels and nerve, to pass through it. This type of connective tissue is important because of its cellular content in the defense against infection and the repair of damaged tissues.

Cells found in the loose connective tissue include, but are not limited to, the following: fibroblasts, which synthesize collagenous connective tissue fibers that are flexible but of great tensile strength; macrophages and monocytes, which ingest, digest, or collect microscopic particles such as debris of dead cells; certain microorganisms; and other nonbiodegradable matter. Mast cells synthesize and release substances of physiological importance (e.g., heparin and histamine).

Dense connective tissue appears in two forms: dense irregular and dense regular connective tissue. The irregular type is found in the dermis of the skin, deep fascia surrounding and defining muscles, capsules of organs and nerve sheaths. Dense regular connective tissue is found primarily in ligaments and tendons and also in ligaments, aponeuroses and the cornea of the eye. While a tendon may be confused with striated muscle at low magnification, the structural differences are easily apparent at higher magnifications. Dense connective tissue contains fewer cells, but, when present, the cells are similar in type to those found in loose connective tissue. Collagenous fibers predominate in dense connective tissue.

Cartilage is a non-vascular tissue containing fibrous connective tissue (collagen Type 2) embedded in an abundant and firm matrix. The cells that produce cartilage are called chondroblasts, and, in mature cartilage where the cells are housed in lacunae, they are termed chondrocytes. Three types of cartilage are recognized: hyaline, elastic, and fibrocartilage. Hyaline cartilage is found at the ventral ends of ribs and in the nose, larynx, trachea, and articular surfaces of adjacent bones of movable joints.

Fibrocartilage is composed predominantly of collagenous (Type 1) fibers arranged in bundles, with cartilage cells surrounded by a sparse cartilage matrix between the fibrous bundles. Fibrocartilage has characteristics similar to both dense connective tissue and hyaline cartilage. It is always associated with dense connective tissue, and, because of its usual paucity of cartilage cells, there appears to be a gradual transition between the two types of connective tissue. Although cartilage cells are not abundant, they are arranged in scattered clusters in parallel arrays, reflecting the direction of stresses placed upon the tissue. Fibrocartilage has no identifiable perichondrium and differs in this regard from hyaline and elastic cartilage. Elastic cartilage is found in the external ear (pinna), auditory tube, epiglottis, and corniculate and cuneiform cartilages of the larynx.

Bone is a tissue that forms the greatest part of the skeleton and is one of the hardest structures of the body. It is the rack upon which all the soft parts are suspended or attached. The skeleton is tough and slightly elastic, withstanding tension and compression. Bone differs from cartilage by having its collagenous connective tissue matrix impregnated with organic salts (primarily calcium phosphate and lesser amounts of calcium carbonate, calcium fluoride, magnesium phosphate, and sodium chloride). The osteoblasts, which form the osseous tissue, become encapsulated in lacunae but maintain contact with the vascular system via microscopic canaliculi. When encapsulated, they are referred to as osteocytes.

Blood and lymph is a type of connective tissue that is peculiar because its matrix is liquid. The blood is carried in blood vessels and is moved throughout the body by the contractile power of the heart. Lymph is found in lymph vessels but originates in extracellular spaces as extracellular fluid, which is normally extravasated from blood capillaries. The extracellular fluid, which enters the lymphatic system of vessels, will have mononuclear white blood cells added to it as the fluid is filtered through lymph nodes, which produce such cells. Lymph is returned to the blood stream near the right and left venous angles (junction of the internal jugular and subclavian veins).

Derived from embryonic mesoderm, mesenchyme is the first connective tissue formed. The cells are widely spaced, with an abundance of intercellular matrix. The primitive mesenchymal cells differentiate into all the supporting tissues of the body. The cells derived from the mesenchyme include blood cells, megakaryocytes, endothelium, mesothelium, reticular cells, fibroblasts, mast cells, plasma cells, special phagocytic cells of the spleen and liver, cartilage cells, and bone cells as well as smooth muscle.

Widely distributed in the embryo as a loose connective tissue, mucoid tissue is composed of large stellate fibroblasts in an abundant intercellular substance, which is homogeneous and soft. In the umbilical cord, it is known as Wharton's jelly.

Muscle cells produce mechanical force by their contraction. In vertebrates there are three main types of muscle. Skeletal muscle moves joints by its strong and rapid contraction. Each muscle is a bundle of muscle fibers, each of which is an enormous multinucleated cell. Smooth muscle is present in digestive tract, bladder, arteries, and veins. It is composed of thin elongated cells (not striated), each of which has one nucleus. Cardiac muscle, intermediate in character between skeletal and smooth muscle, produces the heartbeat. Adjacent cells are linked by electrically conducting junctions that cause the cells to contract in synchrony.

Nerve tissue is specialized tissue making up the central and peripheral nervous systems. Nerve tissue consists of neurons with their processes, other specialized or supporting cells such as the neuroglia, and the extracellular material.

Neuroglia is the supporting structure of nerve tissue. It consists of a fine web of tissue made up of modified ectodermal elements, in which are enclosed peculiar branched cells known as neuroglial cells or glial cells. The neuroglial cells are of three types: astrocytes and oligodendrocytes (astroglia and oligodendroglia), which appear to play a role in myelin formation, transport of material to neurons, and maintenance of the ionic environment of neurons; and microcytes (microglia), which phagocytize waste products of nerve tissue.

The complex biological construct of the subject invention contains at least two subtypes of tissue, each having a different function. The tissues of the complex biological function have diverse function. For example, the complex biological construct may be the paw of an animal having muscle, bones, nerves, skin, connective tissue and hair. In another example, the complex biological construct may be the entire digestive tract of an animal including, but not limited to, muscle tissues from the walls of the stomach and intestine, tissue producing digestive enzymes, and the microvilli of the intestine involved in nutrient absorption.

Isolation of a complex biological construct employs any method of separating and/or severing the construct from an animal. The isolation may be done by surgical procedures on an anesthetized animal including surgical extraction or resection and amputations. Methods resulting in termination of the animal include dissection, severing and excision.

In the preferred embodiment, the complex biological construct is flash frozen with liquid nitrogen immediately after euthanization to maintain the subcellular contents of the construct in the same state as at the time of isolation. Subcellular components include any molecule, macromolecule, or structure present originally within the cell or on the cell surface or which results from the breakage of the cells. Examples include nucleic acids, proteins, metabolites, macromolecular complexes, and desmosomes. Specific proteins may include enzymes, structural proteins, receptors, and signaling proteins. Macromolecular complexes include ribosomes, cytoskeletal fragments, chromosomes, proteosomes, and centromeres.

Flash freezing may be any method where the complex biological construct is completely frozen intact or as a solution or suspension of subcellular components within a few seconds after exposure to cold temperatures. This is generally accomplished by applying extreme cold to the subject via a cryogenic liquid such as liquid nitrogen or dry ice suspended in an alcohol.

Complex biological constructs are tested based on their role in a disease process or their role in a normal function. Problems may arise if only a few cells in the test construct are actively involved in the mechanism or event. Hence, the remaining cells can dilute any signal that could be detected by physical mass alone. For example, 1% of the cells in a tissue give a signal but the remaining 99% mass dilutes the signal to less detectable or nondetectable.

The complex biological construct is then liquefied in lysis buffer (either alone or in combination with a lysis buffer). When the complex biological construct is liquefied, cell lysis occurs. Cell lysis is the rupturing of the cell's plasma membrane and ultimately resulting in the death of the cell. When the cell's plasma membrane is ruptured, the contents of the cell are released. Cell content includes: endoplasmic reticulum responsible for the synthesis and transport of lipids and membrane proteins; mitochondria; cytosol; Golgi apparatus; filamentous cytoskeleton; lysosomes or membrane-bounded vesicles that contain hydrolytic enzymes involved in intracellular digestions; peroxisomes or membrane-bounded vesicles containing oxidative enzymes that generate and destroy hydrogen peroxide; and the cell nucleus.

The cell nucleus stores genes on chromosomes, organizes genes into chromosomes to allow cell division, transports regulatory factors and gene products via nuclear pores, produces messenger ribonucleic acid (mRNA) and organizes the uncoiling of DNA to replicate key genes. The cell nucleus is separated from the cytoplasm by the nuclear envelope. The nuclear contents communicate with the cytosol by means of openings in the nuclear envelope called nuclear pores. The nucleus also has the nucleolus where ribosomes are produced. The nucleolus is organized from the nucleolar organizing regions on different chromosomes. A number of chromosomes transcribe ribosomal RNA at this site.

All of the chromosomal DNA is held in the nucleus, packed into chromatin fibers by its association with histone proteins. Before cell division, the DNA in the chromosomes replicates so each daughter cell has an identical set of chromosome. DNA is responsible for coding all proteins. Each amino acid of DNA is designated by one or more set of triplet nucleotides, code produced from one strand of DNA, by a process called transcription, producing mRNA. mRNA is sent out of the nucleus where its message is translated into proteins. Translation may be done in the cytoplasm on clusters of ribosomes called polyribosomes or on the membranes of the endoplasmic reticulum. The ribosomes provide the structural site where the mRNA sits. The amino acids for the proteins are carried to this site by transfer RNA (tRNA). Each tRNA having a nucleotide triplet that binds to the complementary sequence on the mRNA.

A lysis buffer is a solution containing various components that facilitate cell lysis or cell rupture, and stabilize resulting intracellular components. Examples include detergents, salts, nuclease inhibitors, protease inhibitors, metal chelators such as EDTA and EGTA, lysozyme, and solvents.

The method of the subject invention is especially useful for the extraction and isolation of genetic molecules such as DNA or RNA. The use of a complex biological construct as opposed to a particular tissue sample or organ eliminates the need to analyze the expression patterns in each and every tissue therein to gain an understanding of gene expression patterns within the construct.

In one preferred embodiment of the present invention, the frozen complex biological construct is placed into a sealed chamber along with a liquefying or pulverizing component (herein sometimes referred to as “component”). By the application of force, the liquefying or pulverizing component will disrupt, breakdown and break up the complex biological construct.

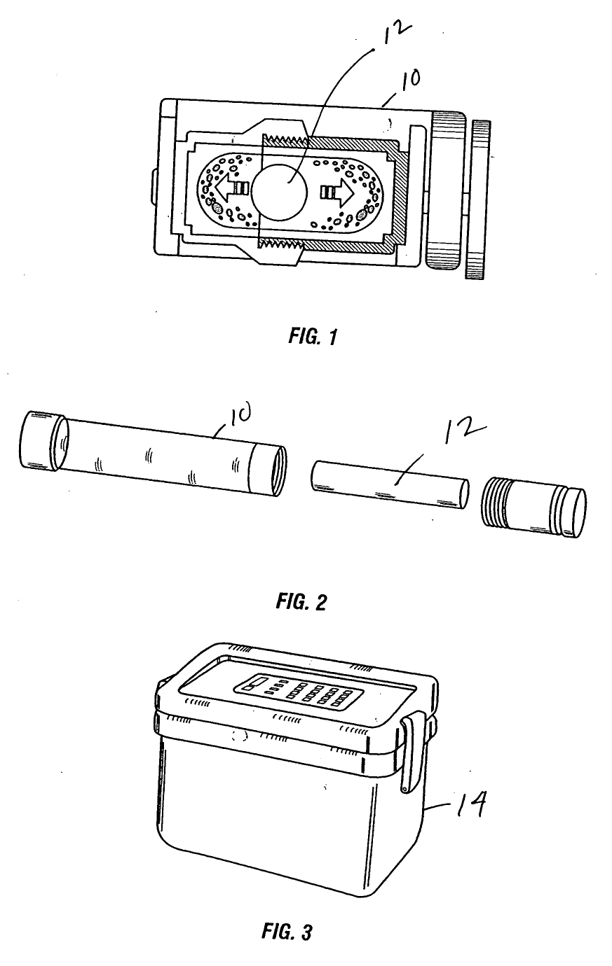

As shown in FIGS. 1 and 2, the apparatus of the preferred embodiment includes a chamber 10 suitable for containing the biological construct and pulverizing or liquefying component 12. The chamber 10 refers to any container designed to hold a complex biological construct. Preferably, the chamber 10 will be of constant shape and diameter in two dimensions to facilitate movement of the component throughout the entire chamber. The chamber 10 may be in the shape of a tube or cylinder, either straight or curved. Preferably, the interior of the chamber 10 will be made of the same material as the component 12 to prevent excessive wear of either the chamber or component 12 from contact of surfaces of varying hardness. The chamber 10 may be made of stainless steel, porcelain glass, chrome steel, agate, or any other appropriate material. Preferably, the interior of the chamber 10 will be made of stainless steel, or, in the case of the freezer mill 14, may be plastic with steel ends.

Suitable chambers include microtube containing small beads, cylinder with closely fitting beads or impactors such as the large cylindrical chamber produce by Retch®, the cryogenic tube-like chambers of the SPEX® CertiPrep 6750 Freezer/Mill 14, and spherical or hemispherical chambers such as that BioSpec® Beadbeater®.

The chamber 10 is designed to facilitate the movement of the liquefying or pulverizing component 12 (as referred to sometimes as a grinding element 12) in and through the chamber 10, or in the case of the freezer mill 14, the tissue moving through a magnetic field which in conjunction with a stainless steel rod within the cylinder powders the tissue. This component 12 may be any object that applies mechanical force or abrasion to the contents of the chamber 10. The component may be a sphere, piston, cylinder closely fitted to the contours of the chamber described above. Alternatively, the component 12 may consist of small beads or sand, a hammer, an abrading surface, or any object capable of crushing, smashing, striking, abrading, compacting, or otherwise bearing on an object.

The component 12 may be considerably smaller than the chamber 10 and thereby capable of free movement therein. Alternatively, the component 12 and chamber 10 may be designed so that the component 12 is shape and size to a cross section of the chamber 10, which is held constant along the length of the chamber 10, thereby allowing lateral movement of the component 12 back and forth across the chamber 10.

A mechanical assembly is provided for imparting motion to either the component 12 or the chamber 10. In a preferred embodiment, the chamber 10 is oscillated, imparting momentum to one or more freely moveable components present therein.

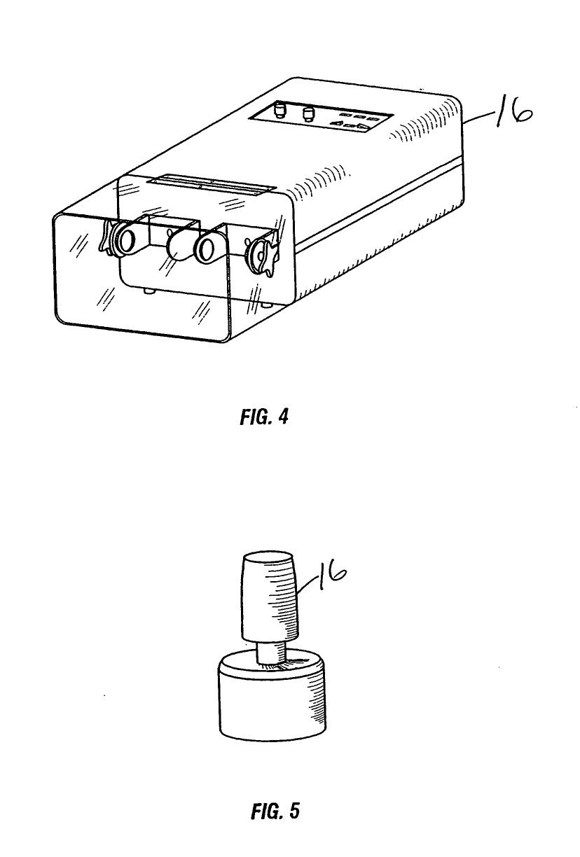

An assembly may be any mechanical device capable of being placed in motion, either manually or by a motor. FIGS. 3 and 4 depict two examples of such assemblies. The assembly may take the form of a mechanical arm, platform, centrifugal device, and magnetically driven impacting devices such as pistons and beads. Oscillatory motion and oscillation refer to any motion that follows a repetitive pattern. Said motion may consist of vibrations, shaking, rocking or swinging. This oscillation may be driven either by applying motion to the grinding element or the assembly itself.

Mechanical force may be applied to the chamber itself to impart momentum to a freely mobile component 12 within the chamber 10, or to the component 12. High speed physical impact of the component 12 on the complex biological construct will result liquefaction or pulverization of the construct, rupture of the cells, and release of intracellular components from the construct.

Devices are currently available in which biological samples are processed into intracellular component through the rapid oscillatory motion of beads, spheres or other objects through a sealed chamber containing the sample. These include the SPEX® CertiPrep 6750 Freezer/Mill, the BioSpec® Beadbeater®, the Retsch® Mixer Mill MM 300, and the Qiagen® Mixer Mill MM 200 (see e.g. FIGS. 3 and 4). Also, as shown in FIG. 5, any type of tissue crusher 18 may be utilized to process the biological sample.

As shown in FIG. 3, the SPEX® CertiPrep 6750 is designed to grind a wide variety of samples including polymers, wood, rubber, and biological tissues. The grinding is carried out at cryogenic temperatures, which provides the advantages of increasing the brittleness of the sample and preventing heat degradation during the grinding process. The grinding itself is vibratory movement of magnetically driven steel impactors through one to four individual grinding chambers. Each grinding chamber 10 or vial is composed of either a polycarbonate or a stainless steel central section with steel endplugs that can withstand the impact of the grinding elements. A magnetic coil drives the motions of the steel impactor and is placed around the chamber. Cryogenic temperatures are maintained by immersing the chambers and coils in liquid nitrogen during the liquefying pulverization since this is only grinding process.

The BioSpec® Beadbeater® is specifically designed for cell disruption. A solid Teflon impeller rotating at high speed forces thousands of minute glass beads to collide with the sample in a specially designed chamber. 90% disruption of the cells can be achieved in less than three minutes.

As shown in FIG. 4, the Retsch® mixer mill 16 is designed as all-purpose grinder capable of processing a large variety of samples ranging from minerals and ores to biological cells. The sample is placed in specially designed chambers made out of a variety of materials including stainless steel, agate, hard porcelain, tungsten carbide, zirconia, and Teflon® along with one or more specially designed balls made out of similar materials. Rapid vibration of the chamber at vibrational frequencies as high as 60 Hz propel the balls through the chamber 10. The disadvantages of the Retsch® mixer mill 16 are it's reliance on the specially designed chambers and the fact that it can only process two chambers at one time if large masses of tissue are used. Forty-eight small tissue samples (2 mg-20 mg) can be processed if an adaptor is used. The Qiagen® mixer mill functions very similarly to Retsch® system but is only designed for the processing of biological samples. The Qiagen® system offers the advantage of being able to process up to 192 samples at the same time using special adaptors that can hold either 96 1.2 ml microtubes or 24 1.5-2.0 ml microtubes. The Qiagen® mixer mill can also process larger sample volumes using the chambers manufactured by Retsch® but like the Retsch® system cannot accommodate more than two such chambers at a time. Qiagen® 3 mm tungsten carbide beads for processing of the smaller samples but similar stainless steel beads can be obtained from either Retsch® or BioSpec®. Like the Retsch® mixer mill, the beads are propelled by rapid vibration of the chamber or tubes, which can be carried out at 3-30 Hz vibrational frequency.

### EXAMPLE 1

### Isolation of RNA from Rat Paws Using Freezer Mill

A rat paw was frozen at −80° C. and placed into stainless steel crusher, pre-chilled at −80° C. Using a hammer, the rat paw was pounded into smaller pieces and transferred to freezer mill grinding vial for two minutes. Freezer mill was filled with liquid nitrogen as per manufacturer's instructions; SPEX CertiPrep® 6750 freezer mill. The tissue was processed for 0.4 minutes and prechilled again for two minutes. The tissue was processed a second time for 0.4 minutes and powdered rat paw was transferred to 50ml conical orange-cap tube and stored at −80° C. until ready to isolate RNA

### EXAMPLE 2

### Rat Paw Isolation Using Mixer Mill

A large stainless steel, screwable cylinders, obtained from Retscho®, catalog #024620169, was placed on dry ice. A large stainless steel grinding balls 20 mm in size, obtained from Retsche® cat #053680062 and #053680070 respectively, were also placed on dry ice.

Two rat paws with toes, nails, and skin were removed from a freezer kept at −80° C. and placed on dry ice immediately. The 20 mm balls were placed in the cooled cylinder half way down. Liquid nitrogen was poured over the rat paws and steel balls and the liquid allowed to bubble off. A cylinder cover is screwed on to the cylinder. The cylinder was then placed onto the Retsch® 200 Mixer Mill (MM 200) for 90 seconds. The cylinder was then removed from the Mixer Mill and then immediately placed on dry ice. Liquid nitrogen was poured over the sample and allowed to bubble off. This process was repeated five more times. One of two rat paws was pulverized. To the pulverized rat paw, 11 ml of 1× lysis buffer (consists of 1 part PBS (without calcium and magnesium) and 1 part ABI lysis buffer) was added and this combination was shaken for three minutes 30 frequency (1/s). This process was repeated four more times. The lysate was then frozen at −80° C.

### EXAMPLE 3

Female Lewis rats (140-150 g) (Harlan Sprague Dawley) were injected ip. with an arthritogenic preparation of Streptococcal Cell Wall (20 ug rhamnose/g animal weight) (Lee Labs). Dosing of the arthritic animals was initiated on day 18. All compounds were dosed po., bid. from day 18-21 except compound #3 which was dosed ip. on day 18 only. At various timepoints between day 18 and 21, animals from the various groups were sacrificed with three animals per treatment group. The rear paws were removed, skinned, and the toes cut off. These paws were flash frozen in liquid nitrogen and stored at −80° C. Frozen paws were precrushed in stainless steel crushers chilled at −80° C. and transferred to freezer mill grinding vials. Tissues were ground in a CertiPrep 6750 freezer mill (SPEX CertiPrep, Inc.) according to manufacturer's instructions by doing 2 rounds of a 2 minute prechill and grinding for 0.4 minutes. The rat paw powder was then stored at −80° C.

RNA was isolated using the Totally RNA Isolation Kit (Ambion, Inc.) according to manufacturer's instructions with the following protocol. 300-400 mg powdered rat paw was added to 6 ml Ambion denaturation buffer and mixed. One starting volume of phenol:chloroform:isoamyl alcohol was then added and the tubes were shaken vigorously for one minute. The tubes were then incubated on ice for 15 minutes followed by a spin at 8,500 rpm at 4° C. in a Beckman JA-17 fixed angle rotor. Following the spin, the top (aqueous) phase was transferred to a new tube and the volume measured. To the aqueous phase was added 1/10 volume of Ambion sodium acetate solution and this was mixed well by inversion. One starting volume of Ambion acid-phenol:chloroform was added and shaken vigourously for one minute. The tubes were incubated on ice for 15 minutes followed by another spin at 8,500 rpm at 4° C. in a Beckman JA-17 fixed angle rotor. Once again the top (aqueous) phase was transferred to a new tube and the volume measured. An equal volume of isopropanol was then added and mixed well. The tubes were incubated at −20° C. overnight and then spun at 8,500 rpm at 4° C. in a Beckman JA-17 fixed angle rotor. Without disturbing the pellet, the supernatant was carefully removed and tubes respun briefly and all residual supernatant removed. Pellets were resuspended in RNase-free water (500 ul -900 ul) and stored at −80° C.

The RNA was further purified by lithium chloride precipitation (Ambion, Inc.) using the following protocol. To 100 μg of RNA that has been resuspended in water, ½ volume Ambion LiCl precipitation solution was added and mixed well by vortexing. This was stored overnight at −20° C. and then spun in a microfuge at 4° C. for 30 minutes at 13,000 rpm. The supernatant was removed and the pellet spun again for a few minutes more. Residual supernatant was removed and the pellet resuspended in 100 μl sterile, RNase-free water (Sigma #W-4502).

Contaminating genomic DNA was then removed by DNasing on RNeasy columns according to manufacturer's instructions (Qiagen). To 100λ of RNA was added 350λ Qiagen RLT containing β-mercaptoethanol buffer and 250 μl 100% ethanol. This was mixed well by pipetting and applied (700 μl) to RNeasy mini-spin column sitting in a collection tube. This was then centrifuged for 15 seconds at >8000×g to bind RNA to membrane. The flow-through and collection tube were discarded and the spin column placed in a new collection tube. To the spin column was added 350 μl Qiagen RW1 buffer and centrifuged for 15 seconds at >8000×g. To each spin column was added 10 μl Qiagen DNase 1 stock mixed in 70 μl Qiagen RDD buffer. This was then incubated at room temperature for 15 minutes. To each spin column was added 350 μl Qiagen RW1 buffer and then spun for 15 seconds at >8000×g. The flow-through was discarded and columns washed with 500 μl Qiagen RPE buffer containing ethanol. Columns were spun again for 15 seconds at >8000 μg and the flow through discarded. This was followed by an additional wash with 500 μl Qiagen RPE buffer and this time the columns were spun for 2 minutes at >8000×g. The columns were then transferred to new microfuge tubes and spun for 1 minute at >8000×g to remove all residual ethanol. The columns were then placed in new microfuge tubes and 30 μl sterile, RNase-free water was added and spun 1 minute at >8000×g to elute the RNA off the column. An additional 30 μl sterile, RNase-free water was added and spun 1 minute at >8000×g. The resulting RNA was stored at −80° C.

The purified RNA was quantitated using a μQuant 96-well spectrophotometer (Bio-Tek Instruments) and a sampling of RNAs were analyzed on a Bioanalyzer 2100 (Agilent Technologies) before TaqMan analysis. Primers and probes for TaqMan analysis were designed using Primer Express Software (Applied Biosystems). Probes were synthesized with the reporter dye FAM at the 5′-end and a non-fluorescent quencher with a minor groove binder at the 3′-end (Applied Biosystems). Taqman reactions were performed using 100 ng total RNA, 500 nM each of forward and reverse primers and 100 nM probe in a 20 μl reaction. 384-well TaqMan plates were pipetted using a BioMek 2000 robot (Beckman Coulter) and TaqMan analysis performed using the 7900HT Sequence Detection System (Applied Biosystems). TaqMan cycling conditions were 48° C. for 30 minutes, 95° C. for 10 minutes, followed by 40 cycles of 95° C. for 15 seconds and 60° C. for 1 minute. Resulting data was analyzed and relative transcription levels calculated using SDS Calculator software (in-house) using normal rat paws as the comparator group.

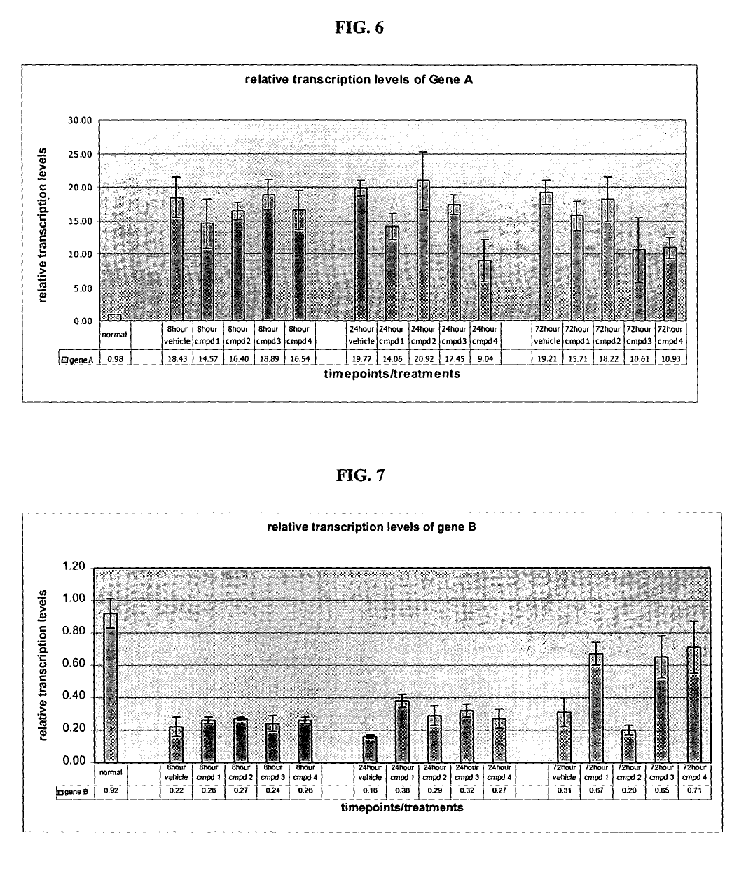

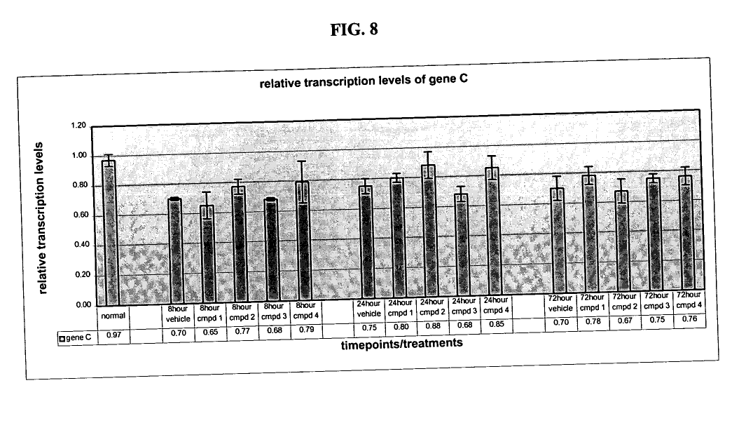

TaqMan analysis was used to assay the panel of RNAs for 31 genes relating to cytokine expression, inflammation, bone formation and degradation and disease modification. Several classes of genes expression were identified. In this animal model, genes were up-regulated (FIG. 6), down-regulated (FIG. 7) or did not change (FIG. 8).

Once the complex biological construct is liquefied and RNA is isolated, genetic expression testing and analysis are prepared. The solution may be frozen prior to running the analysis or used immediately. Genetic molecules may be analyzed by any number of methods well known to those skilled in the art. These methods include hybridization based methods, quantitative or qualitative polymerase chain reaction (PCR), reverse transcriptase PCR (RT-PCR), or real time PCR analysis.

PCR amplification of a specific segment of DNA, referred to as the template, requires that the nucleotide sequence of at least a portion of each end of the template be known. From the template, a pair of corresponding synthetic oligonucleotide primers (“primers”) are designed. The primers will anneal to the separate complementary strands of template, one on each side of the region to be amplified, oriented with its 3′ end toward the region between the primers.

To carry out an analysis using PCR, a known DNA template along with a large excess of two oligonucleotide primers and each deoxyribonucleoside triphosphate, a thermostable DNA polymerase and an appropriate reaction buffer are used. To effect amplification, the mixture is denatured by heat to cause the complementary strands of the DNA template to disassociate. The mixture is then cooled to a lower temperature to allow the oligonucleotide primers to anneal to the appropriate sequences on the separated strands of the template.

Following annealing, the temperature of the reaction is adjusted to an efficient temperature for 5′ to 3′ DNA polymerase extension of each primer into the sequences present between the two primers. This results in the formation of a new pair of complementary strands. The steps of denaturation, primer annealing and polymerase extension can be repeated many times to obtain a high concentration of the amplified target sequence. Each series of denaturation, annealing and extension constitutes one “cycle.” There may be numerous “cycles.” The length of the amplified segment is determined by the relative positions of the primers with respect to each other, and therefore, this length is a controllable parameter. As the desired amplified target sequence becomes the predominant sequence in terms of concentration in the mixture, this sequence is said to be PCR amplified.

With PCR, it is possible to amplify a single copy of a specific target sequence in genomic DNA to a level detectable by several different methodologies. These methodologies include ethidium bromide staining, hybridization with a labeled probe, incorporation of biotinylated primers followed by avidin-enzyme conjugate detection, and incorporation of ³²P-labeled deoxynucleotide triphosphates such as dCTP or dATP into the amplified segment. In addition to genomic DNA, any oligonucleotide sequence can be amplified with the appropriate set of primer molecules. In particular, the amplified segments created by the PCR process are efficient templates for subsequent PCR amplifications leading to a cascade of further amplification. Furthermore, amplification of RNA into DNA can be accomplished by including a reverse transcription step prior to the start of PCR amplification.

By using a single reverse transcription step, one single stranded DNA molecule may be synthesized for each transcript present, thus maintaining the quantitative nature of the procedure. Appropriately controlled quantitative PCR analysis may be performed on the single stranded DNA to obtain an accurate measurement of the amount of a given transcript in the same. These levels may be compared to similar levels of the same transcript in separate samples. In this manner, the transcription of specific transcripts can be correlated with the specific regulatory pathways, giving an indication of the genes involved in the physiological responses regulated by these pathways. Changes in gene expression of one or more genes can indicate their role in a disease process or an individual's inability to metabolize particular therapeutic drugs, i.e., produce toxic side effects, or respond to a particular therapeutic regimen.

Although making and using various embodiments of the present invention have been described in detail above, it should be appreciated that the present invention provides many applicable inventive concepts that can be embodied in a wide variety of specific contexts. The specific embodiments discussed herein are merely illustrative of specific ways to make and use the invention, and do not delimit the scope of the invention. Those skilled in the art will recognize that changes in the apparatus and process may be made without departing from the spirit of the invention. Such changes are intended to fall within the scope of the following claims.

It is to be understood that the disclosed embodiments are merely exemplary of the invention that may be embodied in various and alternative forms. The figures are not necessarily to scale where some features may be exaggerated or minimized to show details of particular components. Therefore, specific structural and functional details disclosed herein are not to be interpreted as limiting, but merely as a basis for the claims and as a representative basis for teaching one skilled in the art to variously employ the present invention.

We claim:

1. A method of analyzing genetic expression comprising the steps of:
liquefying a complex biological construct;
transferring said solution to a microarray; and
determining gene expression.

2. The method of claim 1 wherein the complex biological construct is a gross anatomical structure of an animal comprising more than one type of tissue.

3. A method of analyzing genetic expression comprising the steps of:
placing a complex biological construct into a chamber;
liquefying said complex biological construct in said chamber wherein a solution is formed;
removing said solution from said chamber; and
purifying said solution and extracting and isolating genetic molecules.

4. The method of claim 3 further comprising the step of inserting a component into said chamber wherein said component ruptures the cells of said complex biological component.

5. The method of claim 3 further comprising the step of preparing gene expression analysis.

6. The method of claim 4 wherein said gene expression analysis includes an analysis of gene function.

7. The method of claim 3 wherein genetic molecules are placed in a microarray for matching known and unknown genetic molecules.

8. An apparatus for performing the method of claim 1, comprising:
a component;
a chamber; and
a means for applying force to said chamber wherein said component liquefies the complex biological construct and genetic molecules are release intact.

9. A method of analyzing genetic expression comprising the steps of:
pulverizing a complex biological construct;
transferring said solution to a microarray; and
determining gene expression.

10. The method of claim 9 wherein the complex biological construct is a gross anatomical structure of an animal comprising more than one type of tissue.

11. A method of analyzing genetic expression comprising the steps of:
placing a complex biological construct into a chamber;
pulverizing said complex biological construct in said chamber wherein a solution is formed;
removing said solution from said chamber; and
purifying said solution and extracting and isolating genetic molecules.

12. The method of claim 11 further comprising the step of inserting a component into said chamber wherein said component ruptures the cells of said complex biological component.

13. The method of claim 11 further comprising the step of preparing gene expression analysis.

14. The method of claim 13 wherein said gene expression analysis includes an analysis of gene function.

15. The method of claim 11 wherein genetic molecules are placed in a microarray for matching known and unknown genetic molecules.

16. An apparatus for performing the method of claim 1 comprising:
a component;
a chamber; and
a means for applying force to said chamber wherein said component pulverizes the complex biological construct and genetic molecules are release intact.

17. An apparatus for performing the method of claim 9 comprising:
a component;
a chamber; and
a means for applying force to said chamber wherein said component pulverizes the complex biological construct and genetic molecules are release intact.

18. A method for extraction and isolation of genetic molecules for use in the analysis of genetic expression comprising the steps of
liquefying a complex biological construct into solution having complete and uncontaminated genetic molecules;
transferring said solution to a microarray; and
determining gene expression.

19. The method of claim 18 wherein the complex biological construct is a gross anatomical structure of an animal comprising more than one type of tissue.

20. A method for extraction and isolation of genetic molecules from animal tissue for use in the analysis of genetic expression comprising the steps of:
placing a complex biological construct into a chamber;
liquefying said complex biological construct in said chamber wherein a solution is formed;
removing said solution from said chamber; and
purifying said solution to extract and isolate genetic molecules.

21. The method of claim 20 further comprising the step of inserting a component into said chamber wherein said component ruptures the cells of said complex biological component.

22. The method of claim 20 further comprising the step of preparing gene expression analysis.

23. The method of claim 20 wherein said gene expression analysis includes an analysis of gene function.

24. The method of claim 20 wherein genetic molecules are placed in a microarray for matching known and unknown genetic molecules.

25. A method of extracting genetic molecules from an animal comprising the steps of:
isolating a complex biological construct;
freezing said construct to prevent nucleic acid degradation;
inserting said construct into a chamber fitted with a component wherein said component ruptures the cells of said construct to release genetic molecules and form a solution;
applying force to said chamber;
removing said solution from said chamber wherein said solution contains pure and uncontaminated genetic molecules; and,
freezing said solution for subsequent gene expression analysis.

26. A method of isolating RNA from an animal comprising the steps of:
isolating a complex biological construct;
freezing said complex biological construct to prevent degradation of the RNA;
liquefying said complex biological construct into a solution wherein RNA remains intact; and
freezing said solution prior to purification for subsequent gene expression analysis.

27. An apparatus for reducing a complex biological construct from an animal into solution containing genetic molecules comprising:
a component for rupturing the cells of the complex biological construct and forming a solution;
a chamber for holding said complex biological construct wherein chamber is designed to allow free movement of said component through chamber; and
a means for applying force to said chamber wherein the complex biological construct is liquefied with said component to release genetic molecules intact.

28. An apparatus for performing the method of claim 18, comprising:
a component;
a chamber; and
a means for applying force to said chamber wherein said component liquefies the complex biological construct and genetic molecules are release intact.

29. A method for extraction and isolation of genetic molecules for use in the analysis of genetic expression comprising the steps of
pulverizing a complex biological construct into solution having complete and uncontaminated genetic molecules;
transferring said solution to a microarray; and
determining gene expression.

30. The method of claim 29 wherein the complex biological construct is a gross anatomical structure of an animal comprising more than one type of tissue.

31. A method for extraction and isolation of genetic molecules from animal tissue for use in the analysis of genetic expression comprising the steps of:
placing a complex biological construct into a chamber;
pulverizing said complex biological construct in said chamber wherein a solution is formed;
removing said solution from said chamber; and
purifying said solution to extract and isolate genetic molecules.

32. The method of claim 31 further comprising the step of inserting a component into said chamber wherein said component ruptures the cells of said complex biological component.

33. The method of claim 31 further comprising the step of preparing gene expression analysis.

34. The method of claim 31 wherein said gene expression analysis includes an analysis of gene function.

35. The method of claim 31 wherein genetic molecules are placed in a microarray for matching known and unknown genetic molecules.

36. A method of extracting genetic molecules from an animal comprising the steps of:
isolating a complex biological construct;
freezing said construct to prevent nucleic acid degradation;
inserting said construct into a chamber fitted with a component wherein said component ruptures the cells of said construct to release genetic molecules and form a solution;
applying force to said chamber;
removing said solution from said chamber wherein said solution contains pure and uncontaminated genetic molecules; and,
freezing said solution for subsequent gene expression analysis.

37. A method of isolating RNA from an animal comprising the steps of:
isolating a complex biological construct;
freezing said complex biological construct to prevent degradation of the RNA;
pulverizing said complex biological construct into a solution wherein RNA remains intact; and
freezing said solution prior to purification for subsequent gene expression analysis.

38. An apparatus for reducing a complex biological construct from an animal into solution containing genetic molecules comprising:
a component for rupturing the cells of the complex biological construct and forming a solution;
a chamber for holding said complex biological construct wherein chamber is designed to allow free movement of said component through chamber; and
a means for applying force to said chamber wherein the complex biological construct is liquefied with said component to release genetic molecules intact.

39. An apparatus for performing the method of claim 29, comprising:
a component;
a chamber; and
a means for applying force to said chamber wherein said component pulverizes the complex biological construct and genetic molecules are release intact.
